(19) United States
(12) Patent Application Publication
(10) Pub. No.: US 2004/0265977 A1
(43) Pub. Date: Dec. 30, 2004

(54) Process for the production of scleroglucan

(76) Inventor: Thomas Maier, Schliengen (DE)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/486,756

(22) PCT Filed: Aug. 6, 2002
(86) PCT No.: PCT/EP02/08768

(30) Foreign Application Priority Data
Aug. 15, 2001 (EP) 01810489.6

(51) Int. Cl. C12P 19/04; C12N 1/18

(52) U.S. Cl. 435/101; 435/254.2; 536/123.12

(57) ABSTRACT

A process is described for the production of scieroglucan by culturing the fungus Sclerotium rolfsii ATCC 15205 using a suitable culture medium under microaerobic conditions, wherein the culturing is carried out continuously.

Scleroglucan is an interesting microbial polysaccharide, having a wide variety of possible applications, which is secreted by the filamentous fungus Sclerotium rolfsii during growth. In aqueous solutions, scleroglucan is present as a rod-shaped trimer having a helical structure.

The primary molecular structure may be represented by the following formula:

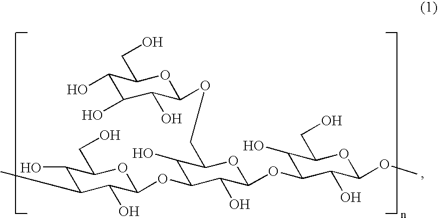

wherein

n is from 9000 to 18 000.

The glucan exhibits strong water-binding properties and is chemically (pH 2-12) and thermally (up to 135° C.) stable.

Scleroglucan is suitable as a viscosifier, while at the same time having wound-healing properties. Further applications are use as a polymeric flooding agent in the context of tertiary crude oil recovery and as a film having low oxygen permeability which is used as a packaging material.

However, the production of glucans by fermentation places high demands on the process technology because of the increase in viscosity during culturing. Numerous processes for glucan production are known which, on the one hand, are very cost-intensive and, on the other hand, provide only unsatisfactory yields.

The problem of the present invention is accordingly to improve, by using new technologies, the overall process of scleroglucan production with respect to economic viability so that it is possible to establish a production process that provides high production yields and meets industrial demands.

It has been found, surprisingly, that the production of scleroglucan can be significantly improved by continuously culturing the fungus Sclerotium rolfsii ATCC 15205 using a suitable culture medium under microaerobic conditions.

The present invention accordingly relates to a process for the production of scleroglucan by culturing the fungus Sclerotium rolfsii ATCC 15205 using a suitable culture medium under microaerobic conditions, wherein the culturing is carried out continuously.

The culturing of Sclerotium rolfsii may be carried out, for example, in stirred bioreactors and stirred chemical reactors.

Reactors suitable for use in the process according to the invention are indicated in the following Table:

TABLE 1
Overview of suitable bioreactors and chemical reactors

| | |
|---|---|
| BR 1 | 48 L bioreactor having a working volume of 30 L, |
| | equipped with 3 four-blade |
| | pitched blade impellers or 3 two-blade Intermig impellers |
| BR 2 | 450 L bioreactor having a working volume of 300 L, |
| | equipped with 3 four-blade |
| | pitched blade impellers |
| BR 3 | 6000 L bioreactor having a working volume of 5400 L, |
| | equipped with 3 six-blade |
| | disk impellers |
| CR 1 | 120 L chemical reactor having a working volume of 95 L, |
| | equipped with 2 or 3 |
| | four-blade pitched blade impellers |

The process according to the invention is preferably carried out in a bioreactor (BR).

The initial pH value in the bioreactor is between 1.5 and 4, preferably between 2 and 3. The temperature in the bioreactor is from 22 to 30° C., preferably from 25 to 28° C. For carrying out continuous culturing, preference is given to the use of the bioreactor BR 1 described in Table 1. After batch-wise culturing (under oxygen-limited conditions), that bioreactor is connected, by way of an intermediate vessel and sterilisable lines, to a storage container (BR 2) containing the culture medium. The culture medium is sterilised beforehand. The intermediate container is used for pressure equalisation. The level of fill is kept constant by means of a float. The specified flow rate of fresh medium is adjusted with the aid of a peristaltic pump.

Further details relating to reactor and process parameters are given in Example 1.

The culture media used comprise the following components, which meet the nutritional needs of the fungus and ensure optimum product formation: glucose, NaNO₃, KH₂PO₄, citric acid, KCl, MgSO₄, FeSO₄, yeast extract, thiamine and ZnSO₄. The media used by way of example, having specific compositions in terms of individual components, are given in the Examples.

Aeration with sterile air is, in each case, carried out through a ring nozzle mounted underneath the impeller system. The reactors are provided with an impeller shaft, driven from below, on which the impellers are mounted. Each reactor usually contains, in addition, four baffles located on opposite sides. In addition, the reactors contain measurement and control devices such as, for example, a temperature control circuit, mass flow meter, speed of rotation control, pH electrode and a sterilisable polarographic oxygen electrode. In addition, the bioreactors may be connected to a waste gas analyser.

The continuous culturing is distinguished by the fact that fresh nutrient solution is continuously fed in and the same amount of culture suspension is discharged. The culturing can be carried out at a constant substrate concentration under constant medium conditions in the transition phase between exponential growth phase (=cell mass increases exponentially over time) and stationary growth phase (=growth stagnates, mass remains constant).

When S. rolfsii is cultured in the continuous process, the product scleroglucan is formed by the biomass present in the bioreactor; the formation of product is coupled to growth, that is to say the formation of product is dependent upon the biomass concentration and the rate of growth.

Depending upon the principle by which the nutrient feed supply is controlled, two types of operation are suitable for the process according to the invention: turbidostat operation and chemostat operation; in the process according to the invention preference is given to chemostat operation.

In a chemostat, the throughflow rate D is controlled, that is to say defined (limited) amounts of chemicals are added per unit of time and, therefore, the growth behaviour is restricted by means of nutrient limitation, which results in a static equilibrium. D also corresponds to the reciprocal of the dwell time of the medium in the reactor.

As a result of substrate limitation, the microorganism is in a state of equilibrium in the transition region between the exponential growth phase and the stationary growth phase. Because a medium constituent is specified in a growth-limited concentration, the chemostat automatically strives against a stable state. As limiting substrate, in many applications, for example, glucose is used as carbon source. When S. rolfsii is cultured under glucose-limiting conditions, however, enzymes (glucanases) are released, which break down the scleroglucan. It is therefore not advisable to use that culturing technique in the case of S. rolfsii. In the process according to the invention, preference is therefore given to nutrient limitation by means of the substrate oxygen. It has been found, surprisingly, that, as a result of the limitation of oxygen, increased secretion of scleroglucan occurs.

In that reaction procedure, the throughflow rate is varied from 0.01 to 0.8 h⁻¹, preferably from 0.04 to 0.13 h⁻¹.

The speed of impeller rotation then ranges from 30 to 300 rpm.

The aeration rate in the bioreactor is between 0.06 and 2.1 V/VM

$$( \frac{\text{volume of air}}{\text{volume of reactor} \cdot \text{minute}} ) .$$

The oxygen partial pressure in the nutrient medium is then reduced from 100% to almost 0%, corresponding to about 10⁻⁴ mol l⁻¹.

Under O₂-limited conditions, the specific glucan productivity is increased (see Example 1a).

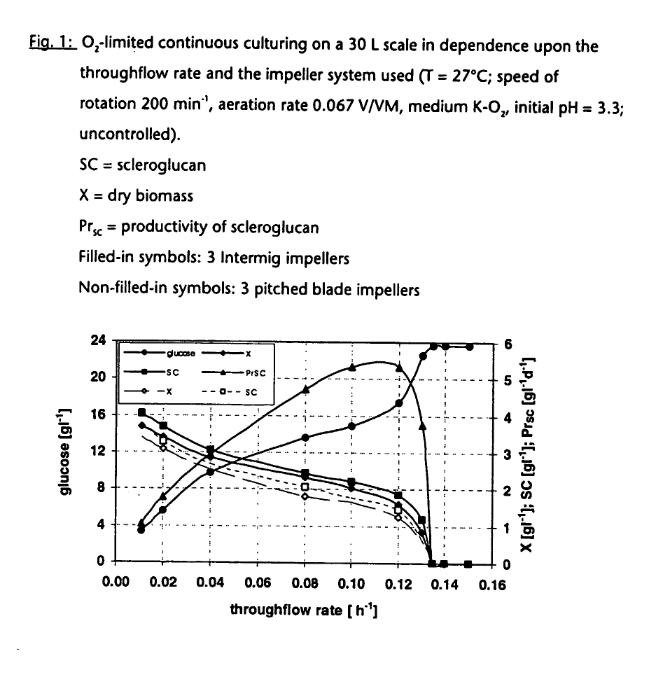

In contrast to carbon-limited continuous culturing, the oxygen-limited chemostat is distinguished by an inversely proportional dependency of the biomass concentration on the throughflow rate, wherein the substrate concentration in the feed supply must not be additionally limiting. For those reasons, a characteristic hyperbolic plot is obtained for the biomass in the X-D diagram of the O₂-limited chemostat (see Example 1a and FIG. 1).

$${C}_{x} = \frac{{Y}_{O} {k}_{L} a}{D} ( {C}_{{O}_{2}}^{*} - \frac{{K}_{O} D}{{μ}_{max} - D} )$$

Cₓ=stationary biomass concentration (g l⁻¹)

D=throughflow rate (h⁻¹)

k_{L}a=volumetric oxygen transport coefficient (h⁻¹)

μₘₐₓ=maximum specific growth rate (h⁻¹)

Yₒ=yield coefficient based on oxygen (−)

Kₒ=substrate saturation constant of oxygen (g l⁻¹)

C*ₒ₂=maximum oxygen solubility (g l⁻¹)

In chemostat operation, besides nutrient limitation by the substrate oxygen, limitation by the substrates nitrogen and/or phosphate is also possible.

In the case of a nitrogen-limited reaction procedure, preference is given to use of an organic and inorganic nitrogen source, the ratio of the organic N source to the inorganic N source ranging from 20:80% to 80:20%. The highest biomass and product concentrations are obtained at a ratio of 80% inorganic nitrogen to 20% organic nitrogen. Preference is given to use of technical-grade yeast extract as the organic nitrogen source and of sodium nitrate as the inorganic nitrogen source.

Potassium dihydrogen phosphate is preferably used as the phosphate source in the case of the phosphate-limited reaction procedure in chemostat operation.

A further process variant according to the invention in the case of the continuous culturing of Sclerotium rolfsii is cell return under oxygen-limited conditions. In that variant, the cells are passed in an external loop over a cross-flow filter by means of an eccentric worm pump, the filtration module having a pore size such that a filtrate that is only just free of cells is obtained. A high flow-over speed in the module of at least 7.5 m s⁻¹ is, however, crucial for an optimum result (no blockage of the membrane).

The return rate R

$$( R = \frac{\text{volume flow of feed supply}}{\text{volume flow of filtrate}} )$$

(or the runback ratio) constitutes one of the most important control parameters in continuous culturing with cell return. The dwell time of the biomass in the system is determined by that variable, and also the dimensions of the volume flows for feed supply, filtrate, retained material and outflow.

In the process according to the invention, the return rate ranges from 0 to 0.95. The highest space-time yields have been achieved at a return rate of 0.95. Preference is given to use of the bioreactor BR 1 for carrying out continuous culturing with cell return. The bioreactor is connected, by way of an intermediate container, to a medium-storage container (BR 2). The specified flow rate of fresh medium is adjusted with the aid of a peristaltic pump. For the purpose of maintaining a constant working volume, a two-channel fill level control means is used. If the liquid volume set is exceeded, a peristaltic pump is triggered by way of a relay and removes the excess of culture broth from the bioreactor in portions. The cell return is connected to the bioreactor in an external loop over a cross-flow filter and a pump. The filtrate contains scleroglucan that is entirely free from cells and runs into a storage vessel. The filtrate container is emptied at regular intervals by means of a pump, controlled by a timer clock.

In the case of continuous culturing with cell return, high space-time yields can be obtained. In conjunction with a long reactor run time, maximum productivities can be achieved. In addition, cell separation is integrated into the production process and can therefore be omitted from the work-up procedure, as a result of which the process as a whole can be configured much more economically and cost-effectively. Also, by appropriately selecting the culturing conditions and cell return system, the high product quality, that is to say a high viscosity yield at low product concentration, as is obtained in the case of batch culturing, need not be lost.

Product work-up is carried out as follows:

1) Oxygen-Limited Chemostat Without Cell Return

The native culture suspension is stabilised against foreign infection by means of formic acid (5 g l⁻¹). The cells are separated out from the undiluted culture suspension by means of cross-flow filtration (sintered metal filter, exclusion limit 10 μm). The cell-free supernatant obtained in that manner is likewise purified with the aid of cross-flow filtration (exclusion limit 0.1 μm) (diafiltration) and/or concentrated. The product solution is, if required, freeze-dried.

2) Oxygen-Limited Chemostat With Cell Return

In this instance, the work-up step of separating out cells is omitted. The cell-free filtrate is purified and, where appropriate, concentrated, in analogous manner to that described above.

The glucans produced in accordance with the invention are suitable as active substances for a very great variety of cosmetic applications such as, for example, in dental-care, denture-care and mouth-care preparations such as, for example, toothpastes, gel toothpastes, tooth powders, mouthwash concentrates, anti-plaque mouthwashes, denture cleaners or denture fixatives, as a formulation for keeping the mucosa moist such as, for example, a vaginal cream or an ophthalmological preparation such as, for example, eye drops, wherein the glucan component may have various functions. Further details relating to the various uses of glucan can be found, for example, in EP-A-0 891 768, page 2, line 14 to page 3, line 41.

The Examples that follow illustrate the invention.

### EXAMPLES

Materials Used

Microorganism

In the following Examples, the fungus Sclerotium rolfsii ATCC 15205, obtained from the American Type Culture Collection, Rockville, Md., U.S.A, is used in scleroglucan production.

Chemicals

In addition to analytically pure chemicals from the companies Fluka (Neu-Ulm) and Boehringer (Mannheim), the following substances are used: potato dextrose agar, Difco Laboratories (Detroit, U.S.A); technical-grade yeast extract, Ohly (Hamburg); Cerelose, Cerestar (Bielefeld) and Ucolub N 115, Brenntag (Munich).

Strain Maintenance

Long-term stocks of Sclerotium rolfsii are stored in liquid nitrogen at −196° C. Storage at −80° C. is possible for a few months under the following conditions. The cells are cultured in StM 3d medium and then suspended in the following solution. 10 g of low-fat milk powder are dissolved in 60 ml of deionised water, 10 ml of glycerol are added dropwise and the solution is then made up to 100 ml with deionised water. After sterilisation, 5 ml of culture suspension are added to 5 ml of the solution and stored at −80° C. The cryotubes are used as inoculum for the pre-cultures.

Pre-Cultures

For the first pre-culture, 100 ml portions of the standard medium StdM (Table 2) are each introduced into a 500 ml baffle-containing Erlenmeyer flask and sterilised. The flasks are then inoculated with the contents (10 ml) of a thawed cryotube and incubated for 100 hours on a rotary shaker in the dark at 27° C. and 100 min⁻.

Zusammensetzung der verwendeten Nährmedien.

TABLE 2
Composition of the used Nutrient media.

| | Indication of the media, data in g l⁻¹ | | | | | |
|---|---|---|---|---|---|---|
| Component | StM | StdM | K—O₂ | K—N | K—PO₄ | K-mZR |
| glucose.H₂O | 38.5 | 38.5 | 10-38.5* | 10-20* | 10-20* | 10-38.5* |
| NaNO₃ | 3.0 | 1.0 | 0.5 | variabel** | 1.0 | 1.0 |
| KH₂PO₄ | 1.3 | 1.3 | 0.7 | 0.7 | variabel** | 1.3 |
| Citric acid.H₂O | 0.7 | 0.7 | 0.35 | 0.35 | 0.35 | 0.7 |
| KCl | 0.5 | 0.5 | 0.25 | 0.25 | 0.25 | 0.5 |
| MgSO₄.7H₂O | 0.5 | 0.5 | 0.25 | 0.25 | 0.25 | 0.5 |
| FeSO₄.7H₂O | 0.05 | 0.05 | 0.025 | 0.025 | 0.025 | 0.05 |
| Technical yeast extract | 1.0 | 1.0 | 0.5 | variable** | 0.5 | 1.0 |
| Thiamine.HCl | 0.0033 | 0.0033 | 0.002 | 0.002 | 0.002 | 0.0033 |
| ZnSO₄.7H₂O | 0.0033 | 0.0033 | 0.002 | 0.002 | 0.002 | 0.0033 |

StM: Strain maintenance media for the preparation of the cryo tube as inoculum
StdM: Standard media
K—O₂: Media for oxygen-limited continuous culturing
K—N: Media for nitrogen-limited continuous culturing
K—PO₄: Media for phosphate-limited continuous culturing
KmZR: Media for oxygen-limited continuous culturing with cell return
*The glucose concentration in the supply it is regulated in such a way that depending upon adjusted flow rate in the expiration a maximum glucose concentration is measured by 8 g l⁻¹.
**the indication “variable” is specified in more detail in the examples.

For the second pre-culture, 500 ml portions of StdM are each introduced into a 2000 ml baffle-containing Erlenmeyer flask and sterilised. Each flask is then inoculated with 5% by volume of inoculum from the first pre-culture. The cultures are incubated for about 75 hours in the dark at 27° C. and 100 min⁻¹.

Nutrient Media

Once all medium constituents have completely dissolved in deionised water, the nutrient solutions are autoclaved for 20 min at 121° C. and 1 bar gauge pressure before being inoculated with Sclerotium rolfsii. The thiamine and ZnSO₄ solutions used are added thereto, sterile-filtered, after inoculation.

### Example 1

### Continuous Culturing

For carrying out continuous culturing, the bioreactor BR 1 described hereinbefore is used exclusively. It is inoculated with 5% by volume of inoculum of the second pre-culture and, after batch-wise culturing for two days (under oxygen-limited conditions), it is connected, by way of a 25 L intermediate container and sterilisable lines, to a 350 L medium storage container (BR 2). The medium contained therein is sterilised beforehand for 20 min at 121° C. and 1 bar gauge pressure, with stirring. The intermediate container serves solely for pressure equalisation. The fill level is kept constant by means of a float. The specified throughflow rate of fresh medium is adjusted with the aid of a peristaltic pump.

The constant working volume of 20 L or 30 L is achieved by means of a two-channel fill level control, which operates according to the conductivity principle on an AC voltage basis (800 Hz). When the level of liquid specified by means of probes is exceeded, a peristaltic pump is triggered by a relay and removes the excess of culture broth in portions from the bioreactor by way of a steam-treated T-piece. In order to ensure problem-free operation, a timer clock allows a maximum continuous pumping time of 5 minutes. When that time is exceeded, for example if the reactor outlet is blocked by biomass, the outflow pump, and also the feed supply pump, are stopped automatically so that, even though the reactor loses its state of static equilibrium, it is not pumped empty or full.

### Example 1a

### Oxygen-Limited Chemostat Having a Variable Throughflow Rate and Variable Aeration Rate

FIG. 1 shows the results of O₂-limited continuous culturing on a 30 L scale in dependence upon the throughflow rate. The throughflow rates are, in each case, varied after establishment of equilibrium, which is achieved after approx. 5-fold volume exchange and which is verified by checking waste gas data measured on-line and using analytical data.

FIG. 1 shows that the biomass exhibits the hyperbolic plot typical of O₂-limited culturing. Because the secretion of scleroglucan is coupled to the growth of biomass, the plot of scleroglucan concentration must correspond, analogously, to the biomass plot. As the throughflow rate increases, both concentrations decrease and approach a critical limit value, the wash-out limit (D_{crtl}=Dₘₐₓ=0.1 35 h⁻¹). When that limit is passed, the biomass is washed out and only substrate remains in the reactor. The productivities increase with an increasing growth rate, passing a maximum of 5.3 g l⁻¹ d⁻¹ at a throughflow rate of D=0.10 h⁻¹. As a result, the optimum throughflow rate for maximum productivity is very close to the wash-out limit. The calculated yield coefficient (quotient of the stationary concentrations of scleroglucan and dry biomass) is, on average, 1.1.

Continuous culturing without cell return should preferably be performed using Intermig impellers. In comparison with pitched blade impellers, stationary biomass and scleroglucan concentrations that are up to 0.5 g l⁻¹ higher, depending upon the throughflow rate, are obtained (FIG. 1).

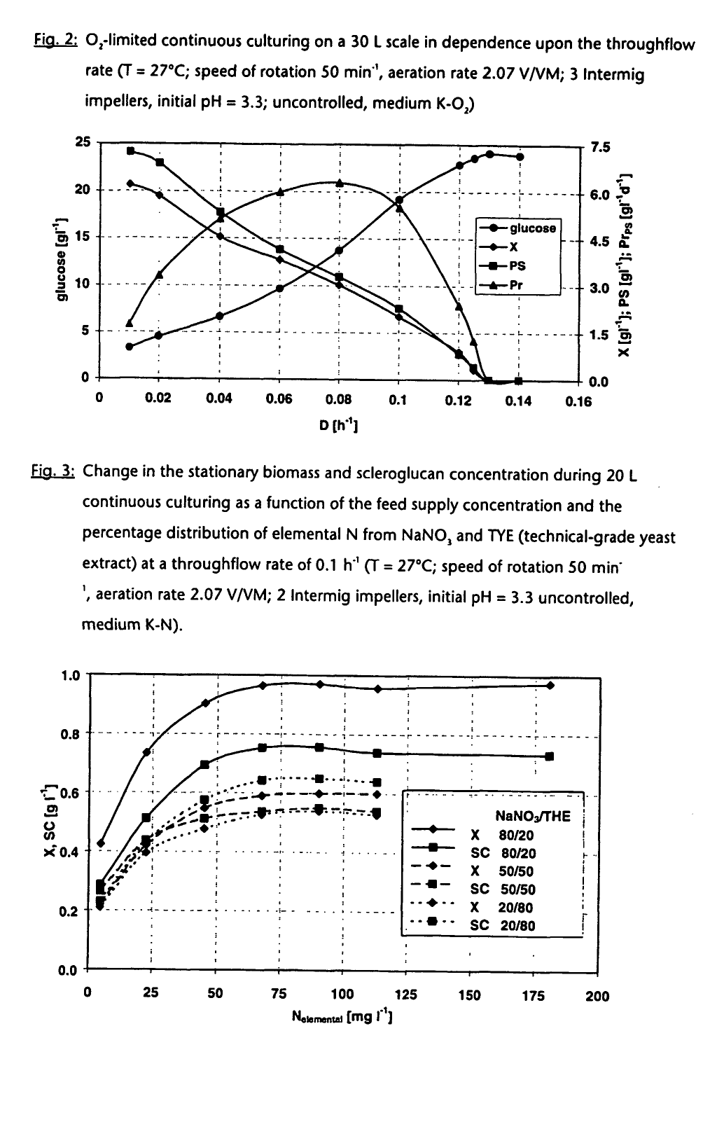

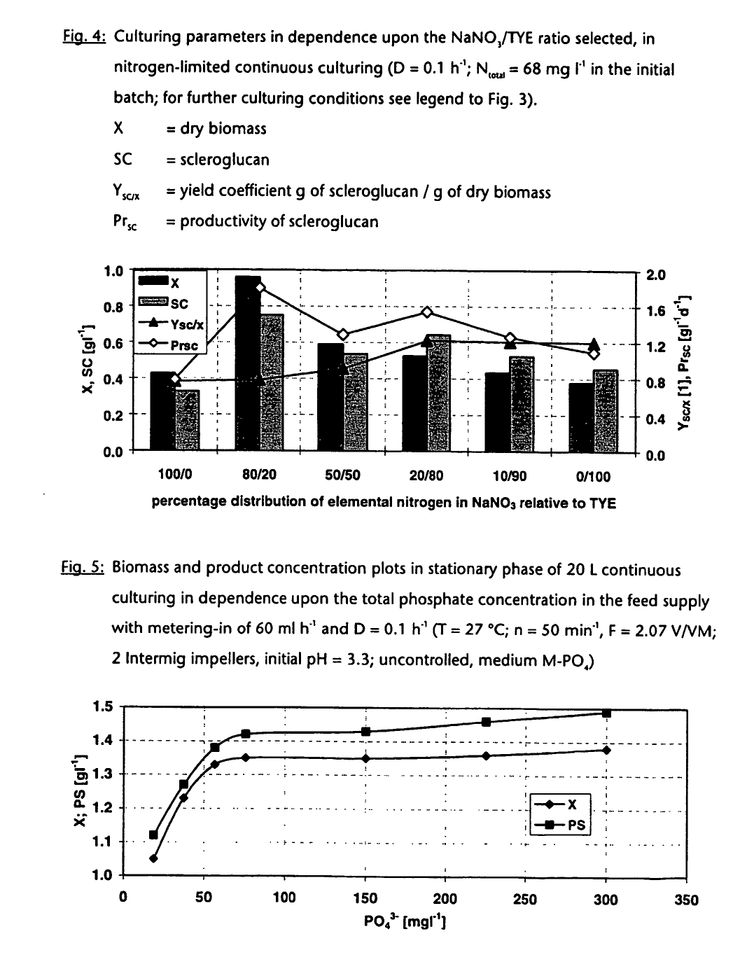

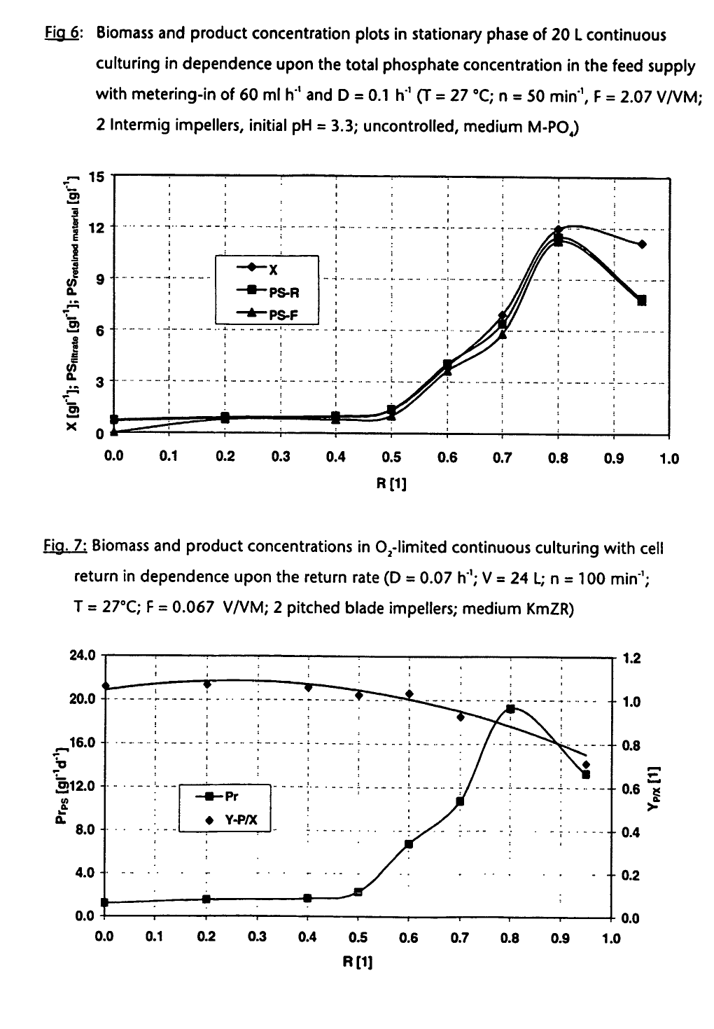

Under the scale-up preconditions of a constant specific power consumption of P/V=0.35 kW m⁻³ and a constant empty-tube gas speed of u_{G}=1.36 cm s⁻¹, the speed of rotation of the Intermig impellers is reduced from 200 min⁻¹ to 50 min⁻¹ and the aeration rate is increased from 0.067 V/VM to 2.07 V/VM, and an X-D diagram is plotted again (FIG. 2). In spite of significantly changed culturing conditions, the results of FIGS. 1 and 2 are similar. FIG. 2 likewise shows strictly growth-coupled product formation. As the throughflow rate increases, both concentrations decrease in this case too and approach the wash-out limit. The yield coefficient is likewise, on average, 1.1. The productivity increases with an increasing throughflow rate although, in contrast to FIG. 1, it passes a maximum of 6.3 g l⁻¹d⁻¹ at a throughflow rate of 0.08 h⁻¹, and it is consequently 18% higher than in FIG. 1 with a lower aeration rate and higher speed of rotation. Even at a throughflow rate of 0.1 h⁻¹, the productivity is still 10% higher.

It is apparent that changing the process parameters speed of rotation and aeration rate improves the culturing results. The energy introduced can be reduced and, as a result, the process becomes more economical. A further advantage of “gentle” mixing at a reduced speed of rotation is an approx. 50% rise in the shear viscosity of the product solution and the increase in quality associated therewith.

### Example 1b

### Nitrogen-Limited Chemostat with Variation of the Nitrogen Source

In this process, the nitrogen concentration must be so selected that the culture is only just nitrogen-limited. At the start, therefore, the nitrogen sources are made ready separately from one another in order to determine the maximum limiting concentration. Starting from low concentrations, the concentration of the limiting substrate in question is increased in stages, and the formation of biomass and product in the individual equilibrium states is investigated. If, at a constant throughflow rate, an increase in the nitrogen concentration in the feed supply results in increased biomass and product formation, then the concentration used beforehand was limiting. If it does not result in an increase, then a different component is already having a limiting action. In order to exclude the possibility of limitation by the nutrient medium components, the medium M-N₂ is used. The possibility of limitation by oxygen can be excluded by means of a prespecified oxygen partial pressure of >10%.

From batch culturing data it can be estimated that 0.165 g of nitrate is required for the formation of 1 g of biomass. That corresponds to an amount of elemental nitrogen of 37.2 mg. The technical-grade yeast extract (TYE) used contains 182 mg of elemental nitrogen per g.

For determination of the maximum permissible amount of nitrogen, operation is carried out at a throughflow rate of 0.1 h⁻¹. The nitrogen-containing solution is metered in separately from the rest of the medium at a throughflow rate of 60 ml h⁻¹ and the resulting difference from the feed supply volume is taken into account. FIG. 3 shows the biomass and scleroglucan concentrations determined in the equilibrium state at different concentrations of sodium nitrate and technical-grade yeast extract. An increase in biomass and scleroglucan is observed up to a concentration of approx. 68 mg l⁻¹ of elemental nitrogen in the feed supply. A further increase in the overall nitrogen concentration does not, in any NaNO₃/TYE concentration ratio, result in an increase in biomass and product formation.

FIG. 4 shows that, without addition of TYE, no acceptable concentrations of biomass and product are obtained. The fungus S. rolfsii is dependent upon organic nitrogen sources such as, for example, amino acids. In the case of culturing wherein sodium nitrate is omitted from the feed supply, similarly unsatisfactory biomass and product concentrations are obtained, which in turn shows that it is likewise not possible to dispense with an inorganic nitrogen source. The highest biomass and product concentration is obtained at a ratio of 80% inorganic nitrogen and 20% organic nitrogen although, in this instance, the yield coefficient has the lowest value of 0.8. If the proportion of sodium nitrate is reduced and the concentration of TYE is increased in stages, the yield coefficient also rises. On the other hand, the maximum achievable final concentrations of biomass and scieroglucan falls. The ratio of 80/20 (NaNO₃/TYE) produces the highest product formation, but with a disproportionately large amount of growth.

### Example 1c

### Phosphate-Limited Chemostat

Phosphate limitation is investigated in a further Example. It is intended to show whether a different nutrient salt component besides nitrogen in a limiting concentration can promote the formation of scieroglucan. For the formation of 1 g of biomass, approx. 69.8 mg of phosphate are required (calculations from batch culturing), which corresponds to an amount of elemental phosphorus of 21.16 mg. The technical-grade yeast extract contains 30 mg of phosphate per g, and phosphate is moreover added to the medium in the form of potassium dihydrogen phosphate.

Continuous culturing is performed at a D of 0.1 h⁻¹ in order that it can be compared with the results from oxygen-limited and nitrogen-limited culturing. The total amount of phosphate (TYE and potassium dihydrogen phosphate) is metered in separately from the rest of the medium at 60 ml h⁻¹ in concentrations of from 0.02 to 0.3 g l⁻¹ and is taken into account in setting the remaining substrate concentration (medium M-PO₄).

FIG. 5 shows the results of phosphate-limited continuous culturing on a 20 L scale at a throughflow rate of D=0.10 h⁻¹. The maximum achievable biomass and product concentrations in dependence upon the amount of phosphate added are shown. Using that graph, in comparison with the phosphate concentrations measured in the supernatant, it is possible to determine the phosphate limitation, resulting in a maximum productivity of 3.4 g l⁻¹d⁻¹ for scleroglucan formation.

The composition of S. rolfsii is known from elemental analysis, according to which it consists of 2.33% nitrogen and 0.43% phosphorus, based on the dry substance. FIG. 5 shows that that amount of phosphorus is likewise required for the formation of 1 g of biomass. The amount of nitrogen required in continuous culturing is greater by a factor of approx. 3 than the amount determined from elemental analysis, but the nitrogen is also required for the formation of secondary products (e.g. glutamine), which have been disregarded here.

### Example 2

### Continuous Culturing with Cell Return

The following Examples are carried out with the aim of adapting a cross-flow filtration system as a cell-retaining system.

The commercially available cross-flow filtration systems (e.g. from Pall and Millipore) offer optimum process control, regulation and monitoring functions and can be readily scaled up. The decisive criterion is the flow-over speed, which must be at least 7 ms⁻¹. All the Examples are carried out using a high-grade steel filter having a pore size of 20 μm. The system is so optimised and technically modified, using valves, seals and high-grade steel lines, that sterile, or monoseptic, operation is possible.

The eccentric worm pump conveys the cells gently at identical throughflow rates so that higher biomass and product concentrations can be achieved. Even though the yields are lower than in the case of continuous culturing where a pump is not included in the apparatus, presumably because of the higher shear stress, it should be possible to increase them by means of cell return and using culturing parameters matched thereto. The higher productivities are achieved using pitched blade impellers.

### Example 3

### Variation of Return Rate at Constant Throughflow Rate

The return rate (or the runback ratio) constitutes one of the most important control parameters in continuous culturing with cell return. That variable determines the dwell time of the biomass in the system and also the dimensions of the volume flows for filtrate, retained material and outflow. Those material flows are, however, dependent not only upon the return rate but also on the throughflow rate. After selecting an optimum filtration module and a low-shear pump, the optimum runback ratio can be determined in continuous culturing using the fungus S. rolfsii at a constant throughflow rate of D=0.07 h⁻¹ on a 24 L scale, with the runback ratio being varied from R=0 to R=0.95. The throughflow rate is reduced from D=0.1 h⁻¹ to D=0.07 h⁻¹ in order to obtain more accurate analyses and results, although that D is not identical to Dₒₚₜ. In addition, the speed of rotation is reduced from 200 min⁻¹ to 100 min⁻¹ so that the entire process is in a state of oxygen limitation.

FIG. 6 shows that, up to a runback ratio of 0.8, the biomass and product concentration increases. Beyond that (R=0.95), a marked drop in product concentration is observed while the biomass is virtually constant.

FIG. 7 shows the productivity and the yield coefficient. The productivity increases to 19.8 g l⁻¹ d⁻¹ at a return rate of R=0.8 and, as such, is higher, by a factor of three, than continuous culturing without cell return. When the concentrations of biomass and scleroglucan obtained and the productivity are compared to continuous culturing without cell return, that is to say when R=0, it can be seen that the concentrations and productivities are significantly lower in the latter case.

### Example 4

### Variation of Throughflow Rate at Optimised Return Rate

The aim is to clarify whether, at higher throughflow rates (D>0.07 h⁻¹), higher return rates and, associated therewith, higher productivities are possible in the case of oxygen limitation or whether an R of 0.8 represents the limit in this system and, consequently, the optimum value. For that purpose, an X-D diagram is plotted for a return rate of R=0.8 and for an R of 0.95. At a return rate of R=0.8 and a maximum growth rate of μ=0.12 h⁻¹, there is a theoretical Dₘₐₓ of 0.6 h⁻¹. At R=0.95, Dₘₐₓ=2.4 h⁻¹ is theoretically possible.

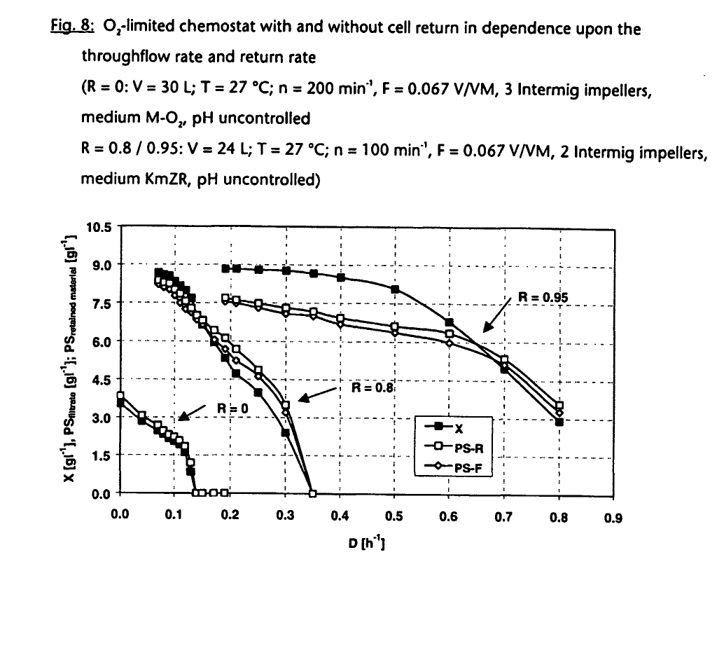

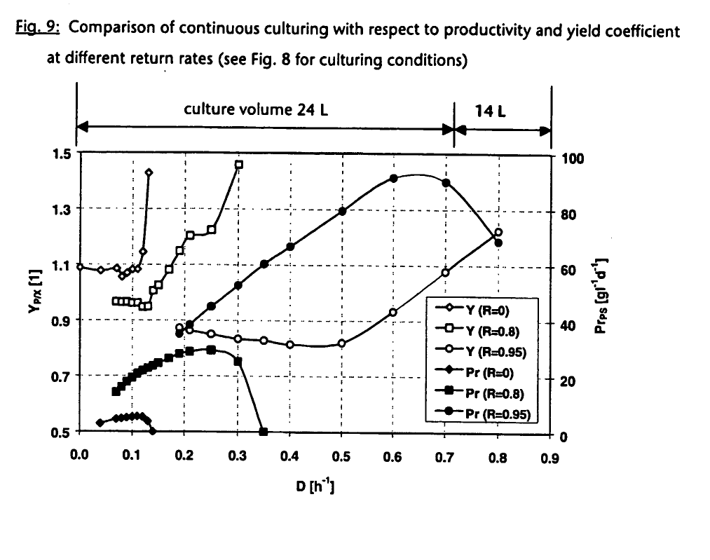

The result of those investigations is shown in FIG. 8, where the biomass in the system and the glucan in the filtrate and retained material are plotted against the throughflow rate.

The data for continuous culturing without cell return (R=0) are taken from FIG. 1. At a return rate of R=0.8, a maximum D of 0.35 h⁻¹ is achieved, which is consequently far below the theoretically determined value of D=0.6 h⁻¹. Maximum productivities of 30 g l⁻¹d⁻¹ are achieved (FIG. 9). Those productivities are higher by a factor of 6.3. In the next step, the extent to which an increase in the return rate to R=0.95 causes a further rise in productivity is investigated. States of equilibrium could be achieved only from a throughflow rate of D=0.19 h⁻¹; below that rate, the high concentration of biomass (>9 g l⁻¹) and product (>7.7 g l⁻¹) make it impossible to operate the filtration system for a prolonged period without blockage of the membrane. From D=0.7 h⁻¹ it was again difficult to maintain a state of equilibrium because the filtration capacity (as much as 16 l h⁻¹ at that point) of the system or the filter area was exhausted. Filtration with pure water resulted in a maximum filtration capacity of approx. 80 l h⁻¹, but in that case the high viscosity and the increasingly thick overlying layer adversely affects the filtration capacity so that the system does not allow higher filtrate flows. A larger filter area or a second module might have provided a solution. Only by reducing the culture volume from 24 L to 14 L was it possible for states of equilibrium to be obtained at a D of 0.7 h⁻¹ or at a D of 0.8 h⁻¹.

The productivity can be increased up to 95 g l⁻¹d⁻¹ , which is higher by a factor of as much as 18 than the productivity in the case of continuous culturing without cell return. With an increasing dilution rate, the respective plots of the yield coefficients rise (FIG. 9), that is to say the glucan yield improves in comparison to biomass formation (FIG. 7). All the tests are carried out with oxygen limitation; the possibility of further limitations, possibly resulting from a lack of substrate, can be excluded by regularly analysing samples of filtrate and retained material for medium constituents such as phosphate, nitrate or glucose.

Continuous culturing with cell return results in average shear viscosities of from 40 to 60 mPas. The higher the throughflow rate selected, the higher the viscosities too. The viscosities are in the same range as batch culturing but, in view of the shear in the filtration module, they are astonishingly high.

TABLE 3
Shear viscosities in dependence upon the throughflow rate at various
return rates

| | | | | | | | | |
|---|---|---|---|---|---|---|---|---|
| R [1] | 0 | 0 | 0.8 | 0.8 | 0.8 | 0.95 | 0.95 | 0.95 |
| D [h⁻¹] | 0.04 | 0.1 | 0.1 | 0.2 | 0.3 | 0.3 | 0.5 | 0.7 |
| η [mPas] | 46.7 | 58.1 | 38.6 | 48.1 | 54.5 | 37.7 | 42.6 | 53.4 |

It is not possible to ascertain any morphological changes compared to batch culturing or to continuous culturing without cell return. The pellets have a diameter of 1-2 mm and are solid. Only on being subsequently cultured in a shake flask do the pellets “grow”, because of the lack of shear, to a diameter of approx. 10 mm. The biomass and product concentration and the productivity after culturing for a period of 72 hours are within the average range of other shake flask culturing procedures.

It can accordingly be demonstrated that the increased shear stress during continuous culturing with cell return has no detectable influence on growth or metabolism.

As a result of increasing the productivity by a factor of 15, at a return rate of R=0.95, compared to continuous culturing without cell return (Prₚₛ=6.3 gl⁻¹d⁻¹) a continuous culturing process can be established by means of which the product scleroglucan can be produced in economically competitive manner. That result is important especially for the design of industrial systems, but it is possible, if a specific productivity is specified, for the bioreactor size to be correspondingly reduced in the case of continuous operation and, accordingly, for procurement costs to be lowered. It is also possible for mixing problems and heat transfer problems to be curtailed. A further advantage of continuous culturing over batch culturing is the time factor lost in starting up and closing down the system and in cleaning.

1. A process for the production of scleroglucan by culturing the fungus Sclerotium rolfsii ATCC 15205 using a suitable culture medium under microaerobic conditions, wherein the culturing is carried out continuously.

2. A process according to claim 1, wherein during the continuous culturing fresh nutrient solution is supplied and, at the same time, the culture suspension is removed.

3. A process according to claim 1, wherein the culturing is carried out in a bioreactor.

4. A process according to claim 3, wherein the pH value in the bioreactor ranges from 1.5 to 4.

5. A process according to claim 3, wherein the temperature in the bioreactor is in the range from 22 to 30° C.

6. A process according to claim 2, wherein the nutrient media are added in defined or limited amounts per unit of time (chemostat operation).

7. A process according to claim 6, wherein in chemostat operation limitation is carried out by the substrate oxygen.

8. A process according to claim 5, wherein the throughflow rate D of the medium is in the range from 0.01 to 0.8 h⁻¹.

9. A process according to claim 6 which is carried out in a bioreactor, wherein the speed of impeller rotation in the bioreactor is from 30 to 300 rpm.

10. A process according to claim 6 which is carried out in a bioreactor, wherein the aeration rate in the bioreactor is in the range from 0.06 to 2.1 VNM (volume of air/volume of reactor·minute).

11. A process according to claim 6 which is carried out in a bioreactor, wherein the oxygen partial pressure in the bioreactor is close to 0%, corresponding to an oxygen concentration of approx. 10⁻⁴ mol/l.

12. A process according to claim 1, wherein the culturing is carried out with cell return.

13. A process according to claim 12, wherein cell return is carried out in a bypass by means of a cross-flow filtration module.

14. A process according to claim 13, wherein the flow-over speed of the filtration module is at least 7 ms⁻¹.

15. A process according to claim 12, wherein the return rate is from 0 to 0.95.

16. A process according to claim 13, wherein no cells are present in the filtrate of the cross-flow filtration module.

17. A process according to claim 12, wherein a low-shear pump system is used.

18. A process for complete separation of fungus mycelium from undiluted culture suspensions by means of cross-flow filtration, wherein a sintered high-grade steel filtration module having a pore size of from 1 to 50 μm is used.

19. A process according to claim 18, wherein the flow-over speed of the filtration module is at least 7 ms⁻¹.

20. A process for the production of inoculum of Sclerotium rolfsii, having reproducible properties in respect of the production of scleroglucan, wherein a Sclerotium rolfsii culture suspension is diluted to 50% using an aqueous solution of 10% low-fat milk powder and 10% glycerol and is frozen at −80° C.
